(19) United States
(12) Patent Application Publication
(10) Pub. No.: US 2008/0050354 A1
(43) Pub. Date: Feb. 28, 2008

(54) PROTECTING BIOACTIVE FOOD INGREDIENTS USING MICROORGANISMS HAVING REDUCED METABOLIZING CAPACITY

(75) Inventor: Peggy GARAULT, Montlhery (FR)

Correspondence Address: FOLEY AND LARDNER LLP;SUITE 500, 3000 K STREET NW, WASHINGTON, DC, 20007, US

(73) Assignee: COMPAGNIE GERVAIS DANONE, Levallois-Perret (FR)

(21) Appl. No.: 11/577,615

(22) PCT Filed: Oct. 17, 2005
(86) PCT No.: PCT/EP05/55309

§ 371 Date: Apr. 20, 2007

(30) Foreign Application Priority Data
Oct. 22, 2004 (FR) 0411277

(51) Int. Cl. A61K 35/74; C12N 1/20

(52) U.S. Cl. 424/93.45; 435/252.3

(57) ABSTRACT

The invention concerns a food product containing living microorganisms and bioactive food ingredients of interest, wherein the living microorganisms and the bioactive food ingredients of interest are organized so as to reduce metabolizing of said bioactive ingredients by said living microorganisms. More particularly, the invention concerns the use therefor of living microorganisms having reduced capacity for metabolizing the bioactive ingredients.

The present invention relates to a food product containing one or more live microorganisms and at least one bioactive food ingredient of interest, in which said live microorganism(s) and said bioactive food ingredient(s) of interest are used so as to reduce the metabolization of said bioactive ingredient(s) by said microorganism(s).

The food ingredients market, in particular of bioactive or functional peptides (i.e. peptides with beneficial activity for the consumer, either locally in the digestive tract, or remotely in the body, after having passed into the circulatory system) has been in full growth for many years.

Bioactive peptides are defined sequences of amino acids that are inactive in their protein of origin, but which have particular properties once released via enzymatic action. They are also known as functional peptides. These bioactive peptides are capable of exerting, inter alia, an effect on the digestive system, the body's defences (for example an antimicrobial or immunomodulatory effect), the cardiovascular system (especially an antithrombotic or antihypertensive effect) and/or the nervous system (such as a sedative and analgesic effect of opiate type) (see tables 1 and 2 below).

Table 1 below lists the main functional peptides released via the hydrolysis of human milk and cow's milk proteins.

TABLE 1

| Original | Functional | Origin of | | Described |
|---|---|---|---|---|
| proteins | peptides* | the milk** | | activities |
| casein α | α-casomorphine | | C | opiate activity |
| | casein α-exorphin | | C | opiate activity |
| | casokinin | | C | antihypertensive |
| | | | | activity |
| casein β | β-casomorphine | H | C | opiate activity |
| | casokinin | H | C | immunomodulatory |
| | | | | activity + |
| | | | | antihypertensive |
| | | | | activity |
| | CPP | H | C | action on |
| | | | | minerals |
| casein κ | CMP = GMP | | C | modulation of |
| | | | | gastrointestinal |
| | | | | motivity and of |
| | | | | the release of |
| | | | | digestive |
| | | | | hormones |
| | casoxine | H | C | opiate |
| | | | | antagonist |
| | casoplatellins | | C | antithrombotic |
| | | | | activity |
| α-lactalbumin | fragments 50-53 | H | C | opiate activity |
| β-lactoglobulin | β-lactorphins | | C | opiate activity + |
| | | | | antihypertensive |
| | | | | activity |
| lactoferrin | lactoferroxin | H | C | opiate |
| lactotransferrin | | | | antagonist |

*the amino acid sequences are not exactly the same
**H: human milk/C: cow's milk

Table 2 below collates the main physiological activities of the functional peptides derived from milk known to date.

TABLE 2

| Activity | Peptides | in vitro | in vivo animal | in vivo man | Ref. |
|---|---|---|---|---|---|
| Effect on | Caseinomacropeptide | Production of CCK by | | | Beucher |
| digestion | (CMP) | rat intestinal cell | | | 1994 |
| | | | Calf: after ingestion of CMP | Man: after ingestion | Yvon 1994 |
| | | | (210 mg/kg), inhibition of | of CMP (4 g), | |
| | | | gastric secretion and | decrease in acid | |
| | | | decrease in plasmatic | secretion | |
| | | | concentration of CKK | | |
| | β-casomorphines | | Rabbit, after introduction | | Ben |
| | | | into the lumen; antisecretory | | Mansour 1988 |
| | | | effect on the ileum | | |
| | | | Dog: after intragastric | | Schusdziarre |
| | | | administration, modulation of | | 1983 |
| | | | postprandial insulinemia; | | |
| | | | cancelling of this effect with | | |
| | | | naloxone | | |
| | Natural β-casomorphines | Several effects on | | | Tome 1987, |
| | and certain | rabbit ileum | | | 1988-Mahe |
| | analogs thereof | | | | 1989 |
| | Unmetabolized | Stimulation of | | | Ben Mansour |
| | β-casomorphine | intestinal absorption | | | 1988 |
| | analogs | of electrolytes | | | |
| | Casein | | Dog: administration of 10 g | | Defillppi |
| | | | casein/300 ml water via | | 1995 |
| | | | intragastric probe: inhibition | | |
| | | | of small intestine motility, | | |
| | | | cancelled with naloxone vs. 10 g | | |
| | | | of soybean protein: no effect | | |
| Anti- | Lactoferricin | Inhibition of growth | | | Tomita 1994-Zucht |
| microbial | Casocidin 1 (α-casein | of pathogenic strains | | | 1995 |
| effect | S₁) - 166-203 | | | | |
| | α-Casein S₁B fragment | Inhibition of growth | Mice, sheep: effective in IM | | Lahov 1996 |
| | (1-23 N terminal) = | of pathogenic strains | injection against | | |
| | isracidin | | Staphylococcus aureus | | |
| | Human β-casein | | Mice: protective effect in IV | | Migliore- |
| | fragment | | injection against K. pneumoniae | | Samour 1989 |
| Immunomodulatory | Fragments of bovine | Proliferation of | | | Kayser |
| effect | α-lactalbumin and of | human lymphocytes | | | 1996 |
| | bovine κ-casein | (PBL) activated with | | | |
| | | Con A | | | |
| | Synthetic β-casokinin | Proliferation or | | | Kayser 1996 |
| | 10 and β-casomorphine 7 | suppression of PBL | | | |
| | | depending on the | | | |
| | | concentration | | | |
| | Human β-casein 54-59 | Stimulation of | | | Parker 1984 |
| | α-lactalbumin 51-53 | phagocytosis of sheep | | | |
| | | red blood cells with | | | |
| | | mouse peritoneal | | | |
| | | macrophages | | | |
| | Bovine β-casein | Stimulation of mouse | No in vivo protection | | Migliore- |
| | 191-193 casein | peritoneal | | | Samour 1988 |
| | 63-68 casein | macrophages | | | |
| | Bovine κ-casein | Inhibition of | | | Otani 1992, |
| | Casein macropeptides | proliferation of B- | | | 1995 |
| | (106-169) | lymphocytes of Peyer | | | |
| | | plaques in mice and | | | |
| | | rabbits | | | |
| Antithrombotic | Bovine caseino- | | | CGP isolated from | Chabance |
| effect | glycopeptide (bCGP) | | | the plasma of | 1995 |
| | Human | | | newborns after | |
| | caseinoglycopeptide | | | ingestion of infant | |
| | (hCGP) | | | milk or mother's | |
| | | | | milk | |
| | Peptide 106-116 of | Inhibition of | | | Jolles 1986 |
| | bovine κ-casein | platelet aggregation | | | |
| | Human | Inhibition of | | | Raha 1988 |
| | Lactotransferrin | platelet aggregation | | | |
| | tetrapeptide (39-42) | | Rat and guinea-pig with | | Drouet 1990 |
| | | | experimental arterial | | |
| | | | thrombosis: after IV | | |
| | | | injection, antithrombotic | | |
| | | | activity | | |
| Antihypertensive | Enzymatic | Inhibition of ACE | | | Mullaly |
| effect | hydrolyzates of | | | | 1997 |
| | β-lactoglobulin and | | | | |
| | of α-lactalbumin | | | | |
| | Synthetic fragments | Inhibition of ACE | Rats receiving angiotensin 1: | | Kohmura |
| | of human β-casein | | after IV injection, return to | | 1989 |
| | | | the initial level of arterial | | |
| | | | pressure | | |
| | Milk peptides | | Hypertensive rats: ingestion | | Masuda |
| | fermented with | | of 10 ml of fermented milk/kg | | 1996 |
| | L. helveticus and | | body weight, the peptides are | | |
| | S. cerevisiae | | found in the aorta with | | |
| | | | inhibition of ACE | | |
| | Peptides derived from | | Hypertensive rats: after | | Yamamoto |
| | milk fermented with | | ingestion, decrease in | | 1994 |
| | L. helveticus | | arterial pressure | | |
| | Peptides derived from | | Hypertensive rats: after | | Nakamura |
| | fermentation of milk | | ingestion, decrease in | | 1995 |
| | with L. helveticus + | | arterial pressure | | |
| | S. cerevisiae Val- | | Normal rats: no effect | | |
| | Pro-Pro [VPP]/II-Pro- | | | Hypertensive humans | Hata 1996 |
| | Pro (IPP) | | | (36 individuals): | |
| | | | | after 8 weeks of | |
| | | | | ingestion of 98 ml/day, | |
| | | | | decrease in | |
| | | | | arterial pressure | |
| Opiate | β-casomorphines | | Rats: after intra-carotid | | Ermisch |
| effects | | | injection, accumulation of β- | | 1983 |
| | | | casomorphines in the blood- | | |
| | | | brain barrier zone | | |
| | | | Newborn calves: after their | | Umbach |
| | | | first meal of cow's milk, | | 1985 |
| | | | β-casomorphines in the blood | | |
| | | | Piglets: after ingestion of | | Meisel 1986 |
| | | | bovine casein, β-casomorphine | | |
| | | | isolated in the duodenal | | |
| | | | chyme | | |
| | | | Puppies: after ingestion of | | Singh 1989 |
| | | | mother's milk, existence of | | |
| | | | β-casomorphines in the blood | | |
| | | | | Man: after ingestion | Svedberg |
| | | | | of cow's milk, | 1985 |
| | | | | presence of β- | |
| | | | | casomorphines in the | |
| | | | | content of the small | |
| | | | | intestine | |
| | | | | but not in the blood | Teschemacher |
| | | | | of adults | 1986 |
| | Synthetic human | Opiate effect on | | | Yoshikawa |
| | β-casein peptides | ileum isolated from | | | 1986 |
| | | guinea-pig, cancelled | | | |
| | | with naloxone | | | |
| | Bovine and human | Antagonist opiate | | | Chiba 1989 |
| | casoxines (κ-casein) | effects on ileum | | | |
| | | muscle isolated from | | | |
| | | guinea-pig | | | |

These peptides are usually obtained by hydrolysis of plant proteins (for example soybean proteins) or animal proteins (for example caseins or whey proteins), the hydrolysis being generated via enzymatic and/or fermentation processes, usually accompanied by concentration of the active fraction, this step generally being necessary to provide the targeted “health benefit”. The manufacture and use of these peptides for health benefit have been the subject of abundant literature (see especially Danone World Newsletter No. 17, September 1998).

Among the food vectors capable of receiving such ingredients, fermented dairy products figure strongly on account of their health benefit due to the presence of ferments and of fermentation products (i.e. molecules derived from the transformation, with lactic acid bacteria, of substrates present in milk). Hitherto, the scientific community took especially into account the properties of ferments. Researchers have very recently begun to show interest in fermentation products, among which certain peptides occupy a particular place, since they are numerous and specific biological messengers. Fermented dairy products thus appear to be particularly suitable as vectors for hydrolyzates of bioactive peptides obtained, for example, from dairy substrates, for instance caseins or whey proteins.

A major problem then arises: the microorganisms, and in particular the lactic acid bacteria, used in the manufacture of fresh dairy products (such as yogurts, fermented dairy specialties, fermented milk-based drinks, etc.) are generally capable of consuming the peptides to satisfy their nutritional requirements, and more particularly their nitrogen requirements. This will be referred to in the text hereinbelow as the “metabolization of peptides”. Specifically, lactic acid bacteria are endowed with several degradation and/or transportation systems allowing them to metabolize peptides, then making them disappear from the medium:

1/ a proteolytic system (PRT wall proteases) that chops up proteins and large peptides to facilitate their assimilation (“extracellular metabolization system”),

2/ systems for transportation into the cell, one of which is specific for oligopeptides of a size close to 10 amino acids, the other being suited to the transportation of dipeptides and tripeptides (lactobacilli have an additional system of tripeptide permeases) (“system(s) for transportation into the cell”), and

3/ an intracellular enzymatic system capable of degrading peptides into amino acids (comprising about 15 endopeptidases and exopeptidases) (“intracellular metabolization system”).

Given that the amount of peptides naturally present in milk is generally too small relative to the needs of lactic acid bacteria, it is common practice to accelerate their growth by providing a supplement of peptides. These are then totally consumed during fermentation.

In summary, on account: (i) of the nitrogen requirement of lactic acid bacteria, of which peptides constitute the main source in milk, (ii) of the capacity of these bacteria to efficiently consume the peptides, and (iii) of the survival of a large population of lactic acid bacteria in fermented milk-based products, up to the expiry date, the use of ingredients based on functional peptides in fermented dairy products is difficult, or even impossible, since these ingredients are usually consumed by the lactic acid bacteria, during fermentation, or even during the storage of the products up to the expiry date.

In addition, not only is this problem of degradation by “untimely” metabolization of peptides by bacteria not specific to a given peptide, it is not specific either to a particular ferment (or microorganism, preferably bacterium, capable of fermenting).

This is a general problem, which arises irrespective of the peptide(s) and of the microorganism(s) under consideration.

Mention will be made, for example, of the case of the bioactive peptide αS₁ [91-100] (see European patent EP 0 714 910; peptide with relaxing properties contained in the milk protein hydrolyzate sold especially by the company Ingredia: 51-53, Avenue Fernand Lobbedez BP 946 62033 Arras Cedex, France, under the name Lactium®). The Applicant has thus observed that the population of live lactic acid bacteria in the finished product continues to metabolize the bioactive peptide during storage of the finished product, to the effect that after only 10 days (for fresh products with an expiry date of 28 days), between 35% and 55% approximately of the bioactive peptide αS₁ [91-100] has disappeared, which is entirely unacceptable for providing the consumer with a “health” effect (data not shown).

Since the consumption of the bioactive peptide is the result of the metabolic activity of ferments, it might be envisioned to reduce this phenomenon by destroying all or some of the microorganisms, for example by means of a suitable heat treatment (thermization or pasteurization). In this case, it is possible to preserve the bioactive peptide αS₁ [91-100] (for example after heating at 75° C. for about 1 minute).

However, such a solution has many drawbacks:
the thermization of a fermented dairy mass entails the use of stabilizers added before the heat treatment (pectins, starches, carrageenans, etc.), which complicates the process and substantially increases the cost of the formula;
the industrial manufacturing line is more complex and requires a greater specific investment;
the product no longer benefits from appellations associated with products containing live ferments (such as yogurt) and as a result looses the benefits associated with the consumption of lactic ferments; and
the generally negative organoleptic impact is significant.

There is consequently a need for a food product containing both live microorganisms, for example a yogurt, and one or more bioactive food ingredients of interest, in which these bioactive food ingredients of interest are protected against metabolization by said live microorganisms, while at the same time preserving the organoleptic qualities of the food product.

With the present invention, the Applicant is providing a solution that can satisfy the existing need.

One subject of the present invention is thus a food product containing one or more live microorganisms and at least one bioactive food ingredient of interest, characterized in that said live microorganism(s) and said bioactive food ingredient(s) of interest are used so as to reduce the metabolization of said bioactive ingredient(s) by said live microorganism(s).

Thus, the Applicant has been able to show that one or more bioactive food ingredients of interest can be efficiently protected against metabolization by live microorganisms, provided that the conditions of use of one with the other are suitable.

Such suitable conditions of use may call upon various means, including:
a) the use of live microorganisms whose capacity to metabolize the bioactive ingredients is reduced; and/or
b) the use of decoy food ingredients that are deliberately “delivered in pasture” to the live microorganisms; and/or
c) the use of a physical protection of the bioactive ingredients, especially by encapsulating them.

It will be noted in this regard that one or more, or even all, of these means may be advantageously combined within the same food product.

As indicated briefly in the preceding general description, the term “metabolized” or “metabolization” is intended to denote, according to the present invention, the transformation or degradation of a substance by one or more live microorganisms, the intention being its consumption as a source of nutrients, and the final consequence being its more or less total disappearance from the medium.

For the purposes of the invention, the metabolization of an ingredient is “reduced” if it is lower than the metabolization of the same ingredient when said ingredient is not protected via at least one of the means provided in the context of the present invention.

Advantageously, and ideally, this reduced metabolization tends towards, or even is, zero, which amounts to little, virtually no, or even no, metabolization of said ingredient.

According to one particular embodiment of the present invention, the residual amount of bioactive food ingredient(s) of interest in said food product is, 3 weeks after its preparation, between about 50% and 100% relative to the amount of bioactive food ingredient(s) of interest present in the product just after its preparation.

Preferentially, said residual amount is between about 80% and 100%.

According to the present invention, the expression “residual amount of bioactive food ingredient(s) of interest in said food product” is intended to denote the percentage of bioactive food ingredient(s) of interest present in said food product when said product is maintained under suitable conditions of storage (for example, from about 4 to 10° C. for a fresh product) for 3 weeks, relative to the percentage of bioactive food ingredient(s) of interest present at the start, i.e. just after manufacture of the product.

According to one particular embodiment of the present invention, said bioactive food ingredient(s) of interest is (are) chosen from:
proteins,
peptides,
analogs or derivatives thereof, and
combinations thereof.

Preferentially, the bioactive food ingredient of interest is chosen from: the bioactive peptide αS₁ [91-100] (see European patent EP 0 714 910), the peptide C6-αₛ₁194-199 (see American patent U.S. Pat. No. 6,514,941), the peptide C7-β177-183 (see American patent U.S. Pat. No. 6,514,941), the peptide C12-αₛ₁23-34 (see American patent U.S. Pat. No. 6,514,941), caseinophosphopeptides, α-casomorphine, α-casein exorphin, casokinin, β-casomorphine, caseinomacropeptides (CMP), also known as glycomacropeptides (GMP) or caseinoglycomacro-peptides (CGMP), casoxine, casoplatellins, fragments 50-53, β-lactorphins, lactoferroxin, the peptides Val-Pro-Pro (see European patent EP 0 583 074), Lys-Val-Leu-Pro-Val-Pro-Gln (see patent application EP 0 737 690), Tyr-Lys-Val-Pro-Gln-Leu (see patent application EP 0 737 690), Tyr-Pro (see patent application EP 1 302 207 and patent EP 0 821 968), Ile-Pro-Pro (see Nakamura et al., 1995; and Japanese patent JP 6 197 786), fragments, analogs and derivatives thereof, proteins and/or peptides containing them, and combinations thereof (for a review, see especially Danone World Newsletter No. 17, September 1998).

Even more preferably, the bioactive food ingredient of interest is chosen from: the bioactive peptide αS₁ [91-100], fragments, analogs or derivatives thereof, proteins and/or peptides containing them, and combinations thereof.

The term “analog” means any modified version of an initial compound, in this case a protein or a peptide, said modified version possibly being natural or synthetic, in which one or more atoms, such as carbon, hydrogen or oxygen atoms, or heteroatoms such as nitrogen, sulfur or a halogen, have been added or removed from the structure of the initial compound, so as to obtain a novel molecular compound.

For the purposes of the invention, a “derivative” is any compound that bears resemblance or has a structural unit in common with a reference compound (protein or peptide). Also included in this definition are, on the one hand, compounds which, alone or with other compounds, may be precursors or intermediate products in the synthesis of a reference compound, by means of one or more chemical reactions, and, on the other hand, compounds that may be formed from said reference compound, alone or with other compounds, via one or more chemical reactions.

The above definition of “derivatives” thus covers at least hydrolyzates, especially trypsin hydrolyzates, of proteins and/or peptides, fractions of hydrolyzates, and also mixtures of hydrolyzates and/or of fractions of hydrolyzates.

Furthermore, the terms “analog” and “derivative of a peptide or protein” mentioned above cover, for example, a glycosylated or phosphorylated peptide or protein or alternatively a peptide or protein that has undergone any grafting of a chemical group.

According to another embodiment of the present invention, the bioactive food ingredient of interest may especially be a sugar or a fatty acid.

Advantageously, said live microorganism(s) is (are) characterized by a reduced, or even zero, capacity for metabolization of the bioactive food ingredients of interest.

According to the present invention, a “reduced capacity for metabolization” is such that the amount of bioactive ingredients of interest metabolized during fermentation (which thus disappears from the medium) is less than or equal to 40% of the initial amount of ingredients (before fermentation).

This is reflected mathematically by:
Qᵣ≧0.6 Qₒ (1)

in which:

Qᵣ: amount of residual bioactive ingredients (present in the medium after fermentation)

Qₒ: initial amount of bioactive ingredients.

The residual amount of bioactive ingredients Qᵣ may be measured via a method of HPLC liquid chromatography coupled to a detector of MS/MS type. An example of an experimental procedure is given in the examples below.

Preferentially, said live microorganism(s) is (are) wild-type strains and/or natural variants and/or mutants obtained via genetic engineering.

In the present patent application, the terms “variant strain” and “variant” are intended to denote a strain obtained mainly by selective mutation from a reference strain, and having the desired property of interest, i.e. the reduced or zero capacity to metabolize said bioactive food ingredient(s) of interest described above.

For the purposes of the invention, a “mutant” or a “mutant strain” is a strain obtained by means of site-directed mutagenesis techniques, from a reference strain. Such a mutant has, like the variants defined above, the desired property of interest.

The techniques for obtaining natural variants by selective mutation, or for obtaining mutants via genetic engineering, especially via genetic transformation using vectors, are known to those skilled in the art. In this regard, reference may be made especially to the publication Sambrook and Russel (2001). In particular, a person skilled in the art may be inspired for the selective mutation protocol from the experimental procedure described by Biswas et al. (1993).

According to one particular embodiment of the present invention, said live microorganism(s) is (are) live bacteria, preferably live lactic acid bacteria.

Preferably, the capacity of at least one mechanism chosen from:
a system for the extracellular metabolization of proteins and peptides,
a system for transporting peptides into the cell, or
a system for the intracellular metabolization of peptides,

is reduced in said live bacteria.

The expression “reduced capacity of a metabolization and/or transport mechanism” denotes herein a metabolization and/or transport mechanism as mentioned above, whose capacity (or activity) does not allow it to metabolize and/or transport more than about 40% of bioactive ingredients of interest during fermentation.

Even more preferably, said mechanism(s) is (are) nonfunctional in said live bacteria.

In particular, said mechanism, of reduced or non-functional capacity, is a system for transporting peptides into the cell.

Even more particularly, said peptide transport system is the AMI system (see, for example, Garault et al., 2002) or the OPP system (in L. bulgaricus: Peltoniemi et al., 2002; in L. lactis: Detmers et al., 1998).

The live bacteria that may be used in the context of the invention may be chosen especially from:
Streptococcus spp, preferably Streptococcus thermophilus;
Lactobacillus spp;
Lactococcus spp; and
Bifidobacterium spp.

Preferably, said live bacteria are chosen from:
Streptococcus thermophilus, deposited at the CNCM (Collection Nationale de Cultures des Microorganismes (Institut Pasteur, Paris, France)) on Jan. 24, 2002 under the number I-2774;
Streptococcus thermophilus, deposited at the CNCM on May 10, 2004 under the number I-3211;
Streptococcus thermophilus, deposited at the CNCM on Sep. 16, 2004 under the number I-3301; and
Streptococcus thermophilus, deposited at the CNCM on Sep. 16, 2004 under the number I-3302.

Even more preferably, said live bacteria are S. thermophilus bacteria deposited at the CNCM on May 10, 2004 under the number I-3211.

Advantageously, the food product according to the present invention contains at least live S. thermophilus and Lactobacillus spp. bacteria.

Preferentially, said live Streptococcus thermophilus bacteria are chosen from S. thermophilus deposited at the CNCM on Jan. 24, 2002 under the number I-2774, S. thermophilus deposited at the CNCM on May 10, 2004 under the number I-3211, S. thermophilus deposited at the CNCM on Sep. 16, 2004 under the number I-3301, and S. thermophilus deposited at the CNCM on Sep. 16, 2004 under the number I-3302.

The content of live microorganisms in the food product according to the invention may vary and will be chosen by a person skilled in the art in the light of his general knowledge in the field. In practice, a standard overall content will preferably be sought, for example of the order of 10⁷ to 10⁹ bacteria per gram of food product.

According to one particular embodiment of the present invention, said bioactive food ingredient(s) of interest is (are) encapsulated.

According to the present invention, the terms “encapsulated” and “encapsulation” are intended to denote the use of a process for protecting an active principle in a vehicle of microparticle type in order to allow a controlled release of this active principle. In the present case, the active principle consists of one or more bioactive food ingredients of interest.

This encapsulation provides a complementary solution to that according to the present invention, in that it allows said bioactive food ingredients of interest to avoid being metabolized by the live microorganisms.

In addition, and entirely advantageously, encapsulation allows a final product that is organoleptically more acceptable to be obtained, for example by masking the more or less strong bitterness of certain bioactive ingredients, in particular of certain peptides.

Finally, encapsulation allows the bioactive food ingredients of interest to reach the intestine without being degraded, and to cross the intestinal barrier unharmed so as to deploy their effects therein.

According to another particular embodiment of the present invention, the food product also contains at least one decoy food ingredient.

According to the present invention, the term “decoy food ingredient” is intended to denote a food ingredient (preferably a peptide, a protein, an analog or derivative thereof, and combinations thereof) capable of serving as a source of nutrients (especially as a source of nitrogen) for live microorganisms, and intended to be preferentially metabolized by said microorganisms, so as to divert said microorganisms from the bioactive ingredients of interest that it is intended, of course, to preserve in priority. Thus, the decoy ingredient represents a nutrient source for the microorganisms, which is deliberately sacrificed in order to maintain the bioactive ingredients of interest as much as possible. The decoy food ingredient acts in this respect as a competitive inhibitor for the transport of the bioactive ingredients of interest.

It will be noted that the particular above-described embodiments may advantageously be combined.

Preferentially, the food product according to the present invention is a fermented product.

Even more preferably, the fermented food product is a dairy or plant product.

According to the present invention, the term “dairy product” is intended to denote, in addition to milk, milk-based products, such as cream, ice-cream, butter, cheese or yogurt; secondary products, for instance whey or casein; and also any prepared food containing as main ingredient milk or milk constituents.

The term “plant product” is intended to denote, inter alia, products obtained from a plant base, for instance fruit juices and plant juices, including soybean juice, oat juice or rice juice.

In addition, the above definitions of “dairy product” and “plant product” each cover any product based on a mixture of dairy and plant products, such as a mixture of milk and fruit juice, for example.

A subject of the present invention is also a process for preparing a food product as described above, in which one or more live microorganism(s) and one or more encapsulated bioactive food ingredient(s) of interest is (are) added to the mixture intended to constitute said food product.

According to one embodiment, said bioactive food ingredient(s) of interest is (are) added to said mixture one after the other.

Alternatively and preferentially, said bioactive food ingredient(s) of interest is (are) added simultaneously to said mixture.

The growing conditions for the microorganisms depend on said microorganisms and are known to those skilled in the art. By way of example, it will be pointed out that the optimum growing temperatures for S. thermophilus are generally between about 36 and 42° C.; they are between about 42 and 46° C. for L. delbrueckii spp. bulgaricus (which is typically found in yogurts).

As a general rule, the stopping of the fermentation, which depends on the pH that it is desired to reach, is obtained by rapid cooling, which allows the metabolic activity of the microorganisms to be slowed down.

According to one particular embodiment of the present invention, said bioactive food ingredient(s) of interest is (are) prepared directly in the mixture intended to constitute said food product. This will be referred to as an in situ synthesis of said bioactive food ingredient(s) of interest.

In the case of an in situ synthesis, it may be envisioned, without preference, that said live microorganism(s) be added to the mixture intended to constitute said food product before, during or after the in situ synthesis of said bioactive food ingredient(s) of interest.

A subject of the present invention is also the use of a food product as described above, as a functional food. The term “functional food” is intended to denote a food product that advantageously affects one or more target functions of the body, independently of its nutritional effects. It may thus result in an improvement in the state of health and/or well-being and/or a reduction in the risks of onset of diseases in a consumer who eats normal amounts of said product. Examples of activities of a “functional food” that will especially be mentioned include anticancer, immunostimulatory, bone-health promoting, antistress, opiate, antihypertensive, calcium-availability enhancing or antimicrobial activities (Functional Food Science in Europe, 1998).

Such functional foods may be intended for man and/or animals.

A subject of the present invention is also the use, in a food product, of a live microorganism with reduced capacity for metabolization of a bioactive food ingredient of interest, to protect said bioactive food ingredient of interest against metabolization by said live microorganism.

The present invention is illustrated by the figures that follow, which are not in any way limiting.

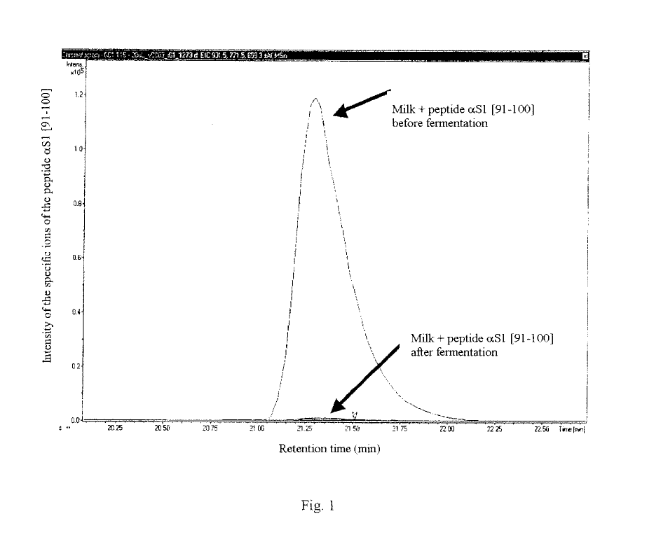

FIG. 1: LC-MS chromatogram illustrating the disappearance of the bioactive peptide αS1 [91-100] included in the ingredient Lactium® during lactic fermentation. The MS/MS detector is regulated so as to reveal only the signal for the m/z ions=634.5 Da (mass of the doubly charged peptide αS1 [91-100]), which produce, after fragmentation, daughter ions of m/z=991.5 Da; 771.5 Da; 658.3 Da (fragments characteristic of the peptide αS1 [91-100]).

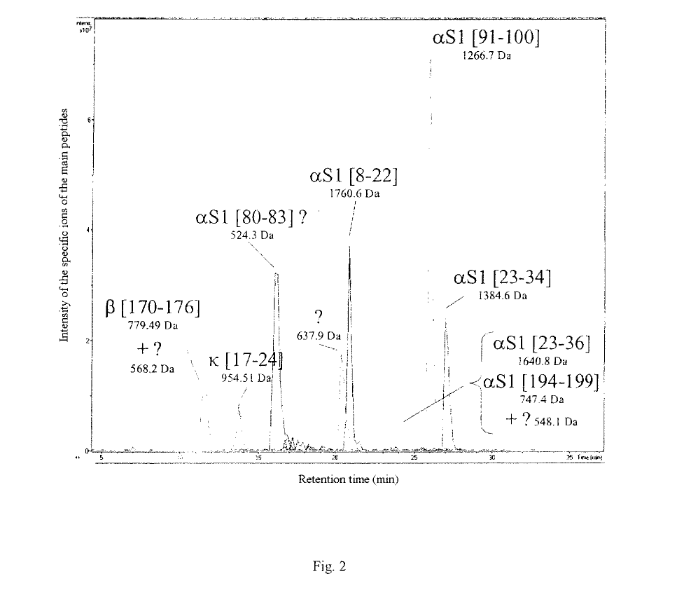

FIG. 2: Identification and quantification of the main peptides of the ingredient Lactium® by LC-MS/MS before and after fermentation of the dairy “mix” with a ferment consisting of a mixture of the strains I-2783 (deposited at the CNCM on Jan. 24, 2002), I-2774 (deposited at the CNCM on Jan. 14, 2002), I-2835 (deposited at the CNCM on Apr. 4, 2002) and I-1968 (deposited at the CNCM on Jan. 14, 1998). After fermentation, these peptides are found only in trace amounts and merge into the base line. “?” means that the identification of the sequence was not possible or is uncertain; only the mass of the peptide is then reported.

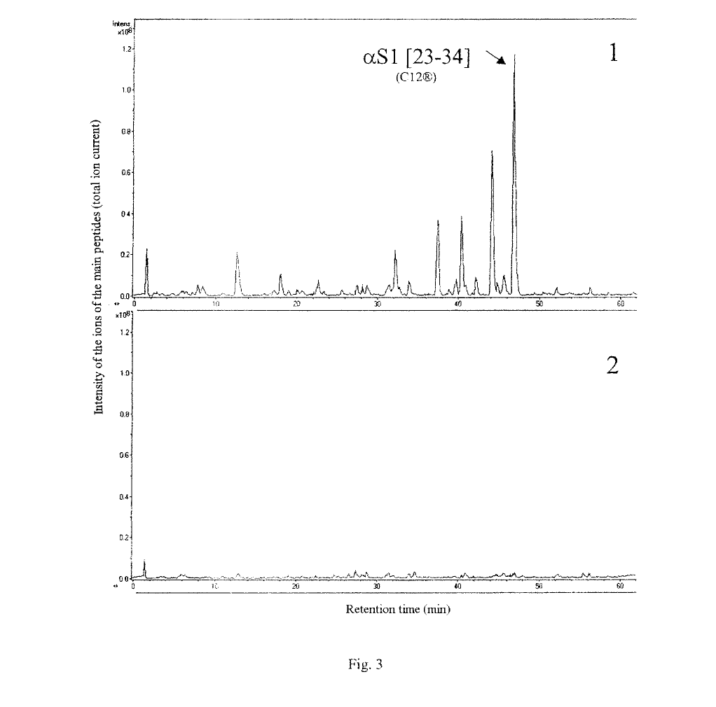

FIG. 3: Compared peptide profiles (LC-MS/MS chromatograms) of a dairy “mix” containing 1.5 g/L of DMV C12® hydrolyzate, before (1) and after (2) fermentation up to pH 4.7 with the lactic ferment Hansen YC380. Virtually all of the peptides of the hydrolyzate, including the bioactive peptide C12 (fragment αS1 [23-34]), have disappeared following metabolization with the strains of the ferment.

FIG. 4: Curves illustrating the change in the residual content of bioactive peptide αS1 [91-100] in a finished product consisting of 95% by mass fermented with the ferment containing the strains I-2783, I-2774, I-2835 and I-1968, and 5% of flavored sugar syrup containing the peptide αS1 [91-100], during storage at 10° C. The experiment was performed in the form of 4 independent tests E1, E2, E3 and E4.

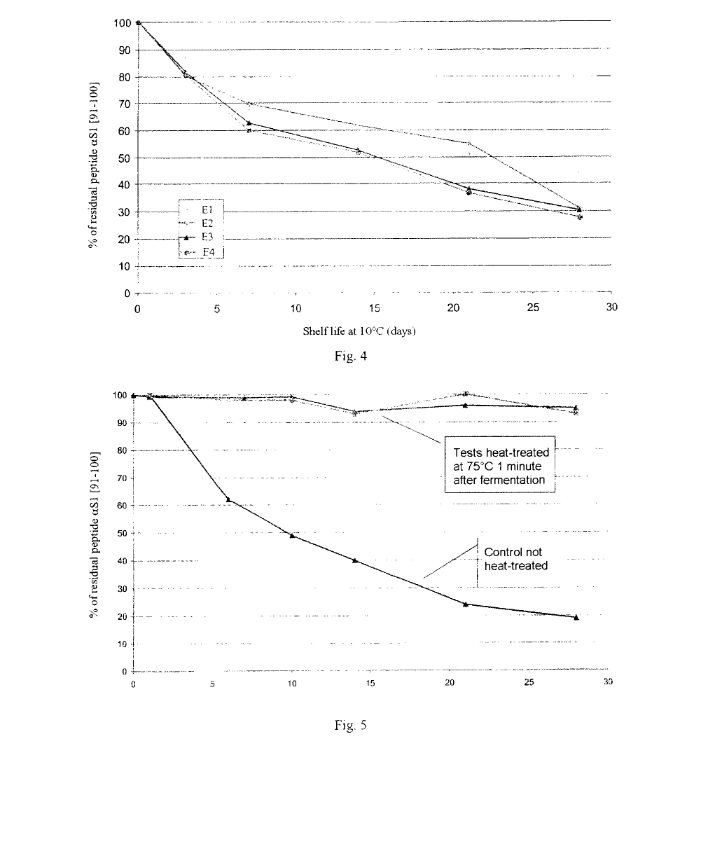

FIG. 5: Curves illustrating the change in the residual content of bioactive peptide αS1 [91-100], added after fermentation in a fermented product and then thermized at 75° C. for 1 minute, and stored at 10° C. up to the expiry date.

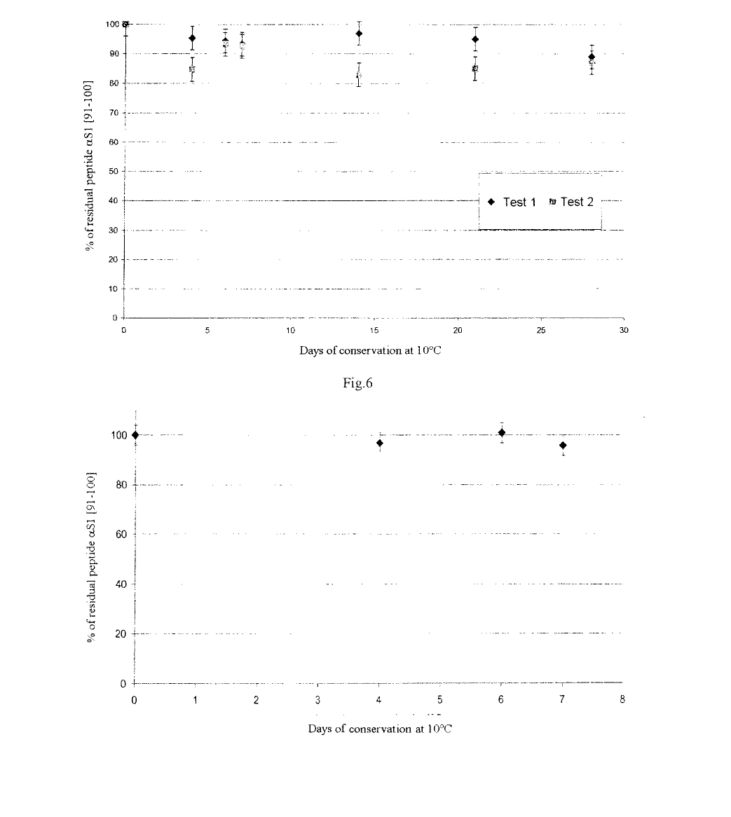

FIG. 6: Illustration of the change in the residual content of bioactive peptide αS1 [91-100] in a finished product consisting of 95% by mass fermented with the ferment containing the strain I-2774 and formate and 5% of flavored sugar syrup containing the peptide αS1 [91-100] (provided in the form of Lactium®, at 1.5 g/Kg of the finished product), during storage at 10° C. up to the expiry date.

FIG. 7: Illustration of the change in the residual content of bioactive peptide αS1 [91-100], added before fermentation, in a finished product consisting of dairy mass fermented with the ferment containing the strain I-2774 and formate.

Other characteristics and advantages of the present invention will emerge on reading the examples that follow, which are given for purely illustrative purposes.

### EXAMPLES

### Example 1

### Use of Bioactive Ingredients of Interest without Applying the Claimed Invention

1.1) Example with the Bioactive Peptide αS1 [91-100] Contained in the Hydrolyzate Lactium®

The use of ingredients of the peptide or protein type, often provided in the form of powders, is simpler when they are added during the step of preparation of the dairy “mix” (powdering of the milk), before the sanitization heat treatment (i.e. 95° C., 8 minutes) and thus before the fermentation. In this case, the risk of metabolization of the active peptide is very high. This is, for example, the case during the use of a functional ingredient such as Lactium® (Ingredia, France) containing a bioactive peptide (fragment 91-100 of casein αS1).

Protocol: the medium was prepared by hydrating a skimmed milk powder at 120 g/L, supplemented with 1.5 g/L of Lactium® ingredient (corresponding to about 30 mg/L of bioactive peptide αS1 [91-100]), and was then pasteurized at 95° C. for 8 minutes.

The lactic ferment was added to a proportion of 0.02%, and the fermentation was performed at the optimum temperature of the selected ferment (between 37 and 42° C.) until a pH of 4.70 is reached.

Analysis of the residual peptides, and especially that of the bioactive peptide αS1 [91-100], was performed via a method of HPLC liquid chromatography coupled to a detector of MS/MS type as described below:
the sample was prepared by diluting the fermented medium in a mixture of water, methanol and trifluoroacetic acid (50/50/0.1%), in a ratio of about 1 to 6. The supernatant after centrifugation constituted the representative sample of the peptide content of the fermented medium.
This sample was injected into an HPLC chromatographic system of Agilent 1100 type (from the company Agilent Technologies France, 1 rue Galvani, 91745 Massy Cedex, France), equipped with a column suitable for peptide analysis, of Waters Symmetry® type (5 μm 2.1×150 mm, WAT056975, Waters France, 5, Rue Jacques Monod, 78280 Guyancourt) at a temperature of 40° C., flow rate of 0.25 ml/min. The peptides were eluted conventionally with an increasing gradient of solvent B (acetonitrile+0.100% formic acid) in solvent A (water+0.106% formic acid), over a period of from 40 minutes to 2 hours as a function of the desired resolution.
The detection was performed using a specific detector of MS/MS type, for example with an ion-trap machine such as Esquire3000+ (Bruker Daltonique, rue de l'Industrie, 67166 Wissembourg Cedex), regulated either for the overall analysis of the peptide content (MS-MS mode), or for the precise and specific quantification of a peptide from its characteristic fragments. For example, the peptide αS1 [91-100] was isolated from its mass (doubly charged ion of mass 634.5 Da) and quantified from the intensity of its characteristic daughter ions after fragmentation (ions of m/z of 991.5 Da, 771.5 Da and 658.3 Da). Even more specifically, an internal standard consisting of the same synthetic peptide deuterated twice (characteristic fragment of 993.5 Da) made it possible to take into account and to set aside any interferences associated with the matrix.

The results are illustrated in FIG. 1.

During its use at this stage (before fermentation with a ferment consisting of a mixture of the strains I-2783 (deposited at the CNCM on Jan. 24, 2002), I-2774 (deposited at the CNCM on Jan. 24, 2002), I-2835 (deposited at the CNCM on Apr. 4, 2002) and I-1968 (deposited at the CNCM on Jan. 14, 1998), or a ferment such as YC380 (Chr. Hansen SA, Le Moulin d'Aulnay, BP64, 91292 ARPAJON Cedex France)), it was demonstrated that more than 95% of the bioactive peptide αS1 [91-100] was consumed after fermentation.

These observations show that the incorporation of bioactive peptides in accordance with the foregoing is not applicable per se to the production of food products, especially dairy products, supplemented with amounts of bioactive peptides and/or proteins that are sufficiently stable over time to be able to observe the desired effect in the consumer.

1.2) Examples with Other Bioactive Peptides of Interest

The results are illustrated by FIGS. 2 and 3.

The ingredient Lactium® contains many other peptides, some of which are potentially biologically active (for instance the fragment 23-34 of casein αS1, which is also sold in the ingredient C12 from the company DMV International). It is interesting to note that virtually all of the peptides provided by adding Lactium® are largely consumed during fermentation.

Irrespective of their origin (originating from various caseins αS1, αS2, κ or β) and their size (from 2 to 3 residues up to 12 residues and more), all the peptides are consumed overall during the fermentation process.

1.3) Use of the Bioactive Peptide αS1 [91-100] (Lactium®) with Other Ferments

In order to check that this phenomenon was not particular to the two ferments used in paragraph 1.1) above, the main industrial ferments, and also various pure strains included in the composition of these ferments, were tested on the basis of the same test: milk reconstituted from powdered milk, to which was added Lactium® at a dose of 1.5 g/L, was fermented under standard conditions (optimum temperature of the ferment between 37 and 42° C., stopping of fermentation at pH 4.7, two repetitions). Analysis of the content of bioactive peptide αS1 [91-100] was then performed on the sample before and after fermentation.

The results obtained on the pure strains are given in Table 3 below:

TABLE 3

| | % of peptide αS1 [91-100] |
|---|---|
| | remaining after |
| | fermentation |
| Pure strains | |
| (S. thermophilus) | |
| I-1630 (Oct. 24, 1995) | 0.3 |
| I-1477 (Sep. 22, 1994) | 0.3 |
| Pure strains (Lactobacillus) | |
| I-1632 (Oct. 24, 1995) | 0.2 |
| I-1519 (Dec. 30, 1994) | 0.1 |
| I-1968 (Jan. 14, 1998) | 1.6 |
| I-2809 (Feb. 19, 2002) | 0.4 |

In Table 3 above, which reflects the consumption of the bioactive peptide αS1 [91-100] by various industrial strains and ferments during the fermentation of a dairy mix containing 1.5 g/L of Lactium®, the pure strains were identified by their respective number and date of deposition at the CNCM (Institut Pasteur, Paris, France).

Table 3 shows that all of the test ferments and strains metabolize from 94% to 100% of the bioactive peptide αS1 [91-100] during the fermentation of a standard dairy mix. The use of this ingredient is thus impossible under standard conditions for producing food products, especially dairy products, containing bioactive peptides and/or proteins in amounts that are sufficiently stable over time to produce an effect in the consumer.

In addition, in order to check that this phenomenon was not particular to the ingredient Lactium®, several combinations of ferments and of other ingredients based on bioactive peptides were studied using the same test (reconstituted milk+test ingredient at a dose of 1.5 g/L, fermented under standard conditions, stopping of fermentation at pH 4.7, two repetitions). The various test combinations are reported in Table 4 below.

TABLE 4

| | Peptide αS1 | Other | | |
|---|---|---|---|---|
| Pure ferments/ | [91-100] in | peptides | DMV | DMV |
| strains | Lactium ® | in Lactium ® | C12 ® | CPP ® |
| Mixture of | X | X | X | X |
| 4 strains: | | | | |
| I-2783 (Jan. 24, 2002) | | | | |
| I-2774 (Jan. 24, 2002) | | | | |
| I-2835 (Apr. 04, 2002) | | | | |
| I-1968 (Jan. 14, 1998) | | | | |
| I-1630 (Oct. 24, 1995) | X | | X | X |
| YC380 Hansen | X | X | X | X |

The ingredients C12 and CPP produced by the company DMV International are milk protein hydrolyzates containing bioactive peptides targeting, respectively, control of hypertension and assimilation of minerals.

On all the experiments, it is seen that all the test ferments have a large capacity for metabolization of the peptides, irrespective of their nature and size.

1.4) Addition After Fermentation

A logical alternative to the procedure studied above is to introduce the functional ingredient after fermentation (process of “delayed differentiation” type), for example with the syrup for flavoring the fermented mass. Use of the same amount of Lactium® ingredient according to this protocol leads to the results illustrated by FIG. 4.

As shown in FIG. 4, even when added under cold conditions (4° C.) after fermentation, the active peptide (provided by the equivalent of 1.5 g of Lactium® per kg of finished product) is rapidly degraded during storage, to leave only 30% to 40% of the initial amount by the expiry date.

Thus, the population of live lactic acid bacteria in the finished product continues to metabolize the bioactive peptide during storage of the finished product, such that after only 10 days (for fresh products whose expiry date is 28 days), between 35% and 50% of the peptide αS1 [91-100] has disappeared, which remains unacceptable for obtaining the desired effect in the consumer.

1.5) Heat Treatment of the Fermented Dairy Product Containing the Bioactive Ingredient of Interest

In this case, it is possible to ensure the stability of the peptide αS1 [91-100] (FIG. 5), but at the expense of the overall quality of the finished product. Specifically, this solution has many drawbacks:
the thermization of a fermented dairy mass entails the use of stabilizers added before the heat treatment (pectins, starches, carrageenans, etc.), which complicates the process and substantially increases the cost of the formula;
the industrial manufacturing line is more complex and requires a larger specific investment;
the product no longer benefits from appellations associated with products containing live ferments (such as yogurt) and as a result loses the benefits associated with the consumption of lactic ferments;
the organoleptic impact (generally negative) is substantial.

### Example 2

### Use of Bioactive Ingredients of Interest by Applying the Claimed Invention

Screening was performed on industrial ferments, on the basis of their capacity not to consume the peptide αS1 [91-100]. Out of the thirty ferments tested, all consume substantially all of the peptide αS1 [91-100] during fermentation, except for one, the ferment containing the only strain I-2774 and formate, the strain I-2774 being microbiologically atypical.

Another strain was tested: it is a natural variant of the strain I-1630 deposited at the CNCM on Oct. 24, 1995 (which, itself, consumes the peptide). This variant I-3211 (deposited at the CNCM on May 10, 2004) was obtained by generation of natural mutants that do not have the peptide transport system (AMI-system). It turns out effectively that this variant does not consume the peptide αS1 [91-100], nor the majority of the other peptides. Among the applications tested at pilot level and even at industrial level, the use of the bioactive peptide αS1 [91-100] supplied in the form of the ingredient Lactium® (Ingredia) was able to be performed successfully by means of using ferment containing the strain I-2774 and formate. FIG. 6 shows the stability results obtained.

Even more spectacularly, the peptide in question was able advantageously to be introduced before fermentation (which simplifies the industrial implementation), as shown in FIG. 7.

Thus, the disappearance of the peptide of interest during fermentation (over a period of 12 hours at ˜41° C.) does not exceed 3% to 4% of the initial amount, whereas the other ferments consume virtually all of the peptides. Under conditions identical to those of the experiments described in Table 4, the strains I-2774 and I-3211 effectively give levels of survival of the peptide αS1 [91-100] after fermentation of greater than 85%.

The greatly reduced capacity for metabolization of the peptides by the strains I-2774 and I-3211 more broadly allows the use of any type of bioactive peptide, including, inter alia, many commercial hydrolyzates that have been the subject of conclusive tests. The experiments described in Table 4 were thus successfully broadened to the use of the ferments/strains I-2774 and I-3211.

### REFERENCES

Kayser et al., (1996) FEBS Letters 383, 18-20

Hata Y. et al., (1996) Am. J. Clin. Nutr. 64, 767-71

Nakamura Y. et al., (1995) J. Dairy Sci. 78, 1253-7

Migliore-Samour D. et al., (1988) Experimentia 44, 188-93

Defilippi C. et al., (1995) Nutr. 11, 751-4

Tomé D. et al., (1987) Am. J. Physiol. 253, G737-44

Tomé D. et al., (1988) Reprod. Nutri. Develop. 28, 909-18

Ben Mansour A. et al., (1988) Pediatr. Res. 24, 751-5

Mahé S. et al., (1989) Reprod. Nutri. Develop. 29, 725-32

Schusdziarra V. et al., (1983) Diabetologia 24, 113-6

Yvon M. et al., (1994) Reprod. Nutri. Develop. 34, 527-37

Zucht H. D., et al., (1995) FEBS Letters 372, 185-8

Tomita M. et al., (1994) Acta Paed. Jap. 36, 585-91

Lahov E. et al., (1996) Food Chem. Toxic. 34, 131-145

Migliore-Samour D. et al., (1989) Int. Dairy Res. 56, 357-62

Jollès P. et al., (1986) Europ. J. Biochem. 158, 379-82

Raha S. et al., (1988) Blood 772, 172-8

Chabance B. et al., (1995) Brit. J. Nut. 73, 582-90

Kohmura M. et al., (1989) Agric. Biol. Chem. 53, 2107-14

Masuda O. et al., (1996) J. Nutr. 126, 3063-8

Yamamoto N. et al., (1994) Biosci. Biotech. Biochem. 58, 776-8

Ermisch A. et al., (1983) J. Neurochem. 41, 1229

Umbach M. et al., (1985) Regul. Pept. 12, 223-30

Singh M. et al., (1989) Pediatr. Res. 26, 34-8

Svedberg J. et al., (1985) Peptides 6, 825-30

Teschemacher H. et al., (1986) J. Dairy Res. 53, 135-8

Yoshikawa M. et al., (1986) Agric. Biol. Chem. 50, 2419-21

Chiba H. et al., (1989) J. Dairy Sci. 72, 363

Beucher S. et al., (1994) J. Nutr. Biochem. 5, 578-84

Parker F. et al., (1984) Eur. J. Biochem. 45, 677-82

Otani H. et al., (1992) Milchwiss. 47, 512-5

Otani H. et al., (1995) J. Dairy Res. 62, 339-48

Drouet et al., (1990) Nouv. Rev. Fr. Hermatol. 32, 59-62

Mullaly M. et al., (1997) Int. Dairy J. 7, 299-303

Meisel H. et al., (1986) FEBS Letters 196, 223-7

Garault et al., (2002) J. Biol. Chem. 277, 32-39

Danone World Newsletter No. 17 (September 1998)

Functional Food Science in Europe (1998) British Journal of Nutrition 80 (1), S1-S193

Biswas et al., (1993) J. Bacteriol. 175 (11), 3628-3635

Sambrook and Russel (2001) Molecular Cloning: a laboratory manual (3rd edition) Cold Spring Harbor Laboratory Press, Cold Spring Harbor, N.Y.

Peltoniemi et al. (2002) Arch. Microbiol. 177(6), 457-67

Detmers et al. (1998) Biochemistry 37(47): 16671-9

1. A food product containing one or more live microorganisms and at least one bioactive food ingredient of interest, characterized in that said live microorganism(s) and said bioactive food ingredient(s) of interest are used so as to reduce the metabolization of said food ingredient(s) of interest by said live microorganism(s).

2. The food product as claimed in claim 1, characterized in that the residual amount of bioactive food ingredient(s) of interest in said food product is, 3 weeks after its preparation, between about 50% and 100% relative to the amount of bioactive food ingredient(s) of interest present in the product just after its preparation.

3. The food product as claimed in claim 2, characterized in that said residual amount is between about 80% and 100% relative to said amount of bioactive food ingredient(s) of interest present in the product just after its preparation.

4. The food product as claimed in any one of claims 1 to 3, characterized in that said bioactive food ingredient(s) of interest is (are) chosen from:
proteins,
peptides,
analogs or derivatives thereof, and
combinations thereof.

5. The food product as claimed in claim 4, characterized in that said bioactive food ingredient(s) of interest is (are) chosen from: the peptide α_{S1} [91-100], the peptide C6-αₛ₁194-199, the peptide C7-β177-183, the peptide C12-αₛ₁23-34, caseino-phosphopeptides, α-casomorphine, α-casein exorphin, casokinin, β-casomorphine, caseinomacropeptides, glycomacropeptides, casoxine, casoplatellins, fragments 50-53, β-lactorphins, lactoferroxin, the peptides Val-Pro-Pro, Lys-Val-Leu-Pro-Val-Pro-Gln, Tyr-Lys-Val-Pro-Gln-Leu, Tyr-Pro, Ile-Pro-Pro, fragments, analogs and derivatives thereof, proteins and/or peptides containing them, and combinations thereof.

6. The food product as claimed in any one of claims 1 to 5, characterized in that said live microorganism(s) has (have) reduced capacity for metabolization of said bioactive food ingredient(s) of interest.

7. The food product as claimed in claim 6, characterized in that said live microorganism(s) is (are) wild-type strains and/or natural variants and/or mutants obtained via genetic engineering.

8. The food product as claimed in any one of claims 1 to 7, characterized in that said live microorganism(s) is (are) live bacteria, preferably lactic acid bacteria.

9. The food product as claimed in claim 8, characterized in that the capacity of at least one mechanism chosen from:
a system for the extracellular metabolization of proteins and peptides,
a system for transporting peptides into the cell, or
a system for the intracellular metabolization of peptides,
is reduced in said live bacteria.

10. The food product as claimed in claim 9, characterized in that said mechanism is nonfunctional in said live bacteria.

11. The food product as claimed in claim 9 or 10, characterized in that said mechanism is a system for transporting peptides into the cell.

12. The food product as claimed in claim 11, characterized in that said peptide transport system is the AMI system or the OPP system.

13. The food product as claimed in any one of claims 8 to 12, characterized in that said live bacteria are chosen from:
Streptococcus spp, preferably Streptococcus thermophilus;
Lactobacillus spp;
Lactococcus spp;
Bifidobacterium ssp.

14. The food product as claimed in claim 13, characterized in that it contains at least live S. thermophilus and Lactobacillus spp bacteria.

15. The food product as claimed in claim 13 or 14, characterized in that said live S. thermophilus bacteria are chosen from:
Streptococcus thermophilus, deposited at the CNCM (Collection Nationale de Cultures des Microorganismes (Institut Pasteur, Paris, France)) on Jan. 24, 2002 under the number I-2774;
Streptococcus thermophilus, deposited at the CNCM on May 10, 2004 under the number I-3211;
Streptococcus thermophilus, deposited at the CNCM on Sep. 16, 2004 under the number I-3301; and
Streptococcus thermophilus, deposited at the CNCM on Sep. 16, 2004 under the number I-3302.

16. The food product as claimed in claim 15, characterized in that said live bacteria are S. thermophilus bacteria deposited at the CNCM on May 10, 2004 under the number I-3211.

17. The food product as claimed in any one of claims 1 to 16, characterized in that said bioactive food ingredient(s) of interest is (are) encapsulated.

18. The food product as claimed in any one of claims 1 to 17, characterized in that it also contains at least one decoy food ingredient.

19. The food product as claimed in any one of claims 1 to 18, characterized in that it is a fermented product.

20. The food product as claimed in claim 19, characterized in that it is a dairy or plant product.

21. A process for preparing a food product as claimed in any one of claims 1 to 20, characterized in that said live microorganism(s) and said bioactive food ingredient(s) of interest is (are) added one after the other to the mixture intended to constitute said food product.

22. A process for preparing a food product as claimed in any one of claims 1 to 20, characterized in that said live microorganism(s) and said bioactive food ingredient(s) of interest is (are) added simultaneously to the mixture intended to constitute said food product.

23. A process for preparing a food product as claimed in any one of claims 1 to 20, characterized in that said bioactive food ingredient(s) of interest is (are) prepared directly in the mixture intended to constitute said food product.

24. A process for preparing a food product as claimed in claim 23, characterized in that said live microorganism(s) is (are) added to the mixture intended to constitute said food product, before the in situ synthesis of said bioactive food ingredient(s) of interest.

25. A process for preparing a food product as claimed in claim 23, characterized in that said live microorganism(s) is(are) added to the mixture intended to constitute said food product, during the in situ synthesis of said bioactive food ingredient(s) of interest.

26. A process for preparing a food product as claimed in claim 23, characterized in that said live microorganism(s) is(are) added to the mixture intended to constitute said food product, after the in situ synthesis of said bioactive food ingredient(s) of interest.

27. The use of a food product as claimed in any one of claims 1 to 20, as a functional food.

28. The use, in a food product, of a live microorganism with reduced capacity for metabolization of a bioactive food ingredient of interest, for protecting said bioactive food ingredient of interest against metabolization by said live microorganism.
